(19) United States
(12) Patent Application Publication
(10) Pub. No.: US 2010/0125267 A1
(43) Pub. Date: May 20, 2010

(54) Plasma Gun for Bio/Medical Treatment

(75) Inventors: Keun Ho Lee, Seongnam-si (KR); Hae Ryong Lee, Gyeonggi-do (KR); Jung Mi Hong, Gyeonggi-do (KR); Yong Nam Choi, Gyeonggi-do (KR); Mun Sup Choi, Gyeonggi-do (KR)

Correspondence Address: WOODCOCK WASHBURN LLP, CIRA CENTRE, 12TH FLOOR, 2929 ARCH STREET, PHILADELPHIA, PA, 19104-2891, US

(73) Assignee: PSM INC., Gyeonggi-do (KR)

(21) Appl. No.: 12/325,817

(22) Filed: Dec. 1, 2008

(30) Foreign Application Priority Data
Nov. 14, 2008 (KR) 10-2008-0113272

(51) Int. Cl. A61B 18/04; H01J 7/24

(52) U.S. Cl. 606/27; 315/111.21

(57) ABSTRACT

There is disclosed a plasma gun for bio/medical treatment using atmospheric plasma. The disclosed plasma gun comprises a housing having an elongate chamber provided therein, the chamber having an end at which a nozzle for spraying plasma is positioned; a gas supply unit for supplying a reaction gas to the chamber; and a plasma discharge unit formed with an elongate cavity communicating with the nozzle, the plasma discharge unit including first and second electrodes and a dielectric or insulating barrier material for plasma ignition in the elongate cavity.

### CROSS-REFERENCE

This application claims priority to Korean Application No. 10-2008-0113272, filed Nov. 14, 2008, which is hereby incorporated by reference in its entirety

### TECHNICAL FIELD

The present invention relates to a bio/medical apparatus, more particularly, to a plasma gun for medical treatment using atmospheric plasma, and still more particularly, to a plasma gun for medical treatment having the configuration suitable for medical applications such as healing for injuries, sterilization for biological tissues, treatment for blood coagulation, healing for skin tumors, healing for bone tumors, fat management and treatment, and skin management and operation

### BACKGROUND

Plasma having electrons, ion species, and neutral active species with high energy has been employed in various applications for a long time due to an effective surface treatment mechanism. As the large-sized flat panel display industry is grown, there is a need for apparatuses that process a workpiece in line under atmospheric pressure instead of using plasma conventionally generated under a vacuum condition, and a technique using atmospheric plasma substitutes conventional vacuum equipments with the apparatuses operating under atmospheric pressure, whereby the technique using atmospheric plasma is gradually employed in various applications.

Even in bio/medical fields, there have been many efforts to utilize plasma capable of various surface treatment depending on types of reaction gases, power source properties and the like, and attempts have recently been realized to apply the plasma more safely and effectively.

For example, U.S. Patent Application Publication No. US2007/0029500 filed and published with the title of “Plasma Source and Applications thereof” discloses an apparatus, which comprises a plasma forming region, a plasma excitation region and a plasma exit and can be used in the limited treatment applications such as healing for skin and removal of damaged skins. The disclosed apparatus is configured so that an activated electrode having a capillary structure passes into a tube, which is provided with an exit and serves as a ground electrode, and a reaction gas is supplied into the tube.

Since the aforementioned conventional apparatus has a problem in that there is no sufficient allowance for time during which the reaction gas is ignited, it has been difficult to allow the plasma to be stably and reliably generated. Further, since a gap between the activated electrode and the ground electrode is fixed, there is a problem in that the degree of freedom in design is deteriorated. Furthermore, since the thin and elongate tube having the exit serves as a nozzle, the conventional apparatus has no option but to allow the plasma to exit in the form of a point having a small fixed area, which makes it impossible to vary the operational area of the plasma and to realize various medical applications. Still furthermore, since the power source in the conventional apparatus is limited to a radio frequency (RF) power source, there is a problem in that a type of the reaction gas which may be used in the conventional apparatus is limited.

### SUMMARY

Accordingly, an object of the present invention is to provide a plasma gun for medical treatment, wherein an improved configuration of a chamber supplied with a reaction gas and a plasma discharge unit independently positioned within the chamber causes the plasma to be stably and reliably generated, so that the generated plasma may be used as medial applications for biological tissues, and wherein various nozzles capable of adjusting the area (and shape) of the plasma may be employed.

According to an aspect of the present invention, there is provided a plasma gun for medical treatment using atmospheric plasma, which comprises a housing having an elongate chamber provided therein, the chamber having an end at which a nozzle for spraying plasma is positioned; a gas supply unit for supplying a reaction gas to the chamber; and a plasma discharge unit formed with an elongate cavity communicating with the nozzle, the plasma discharge unit including first and second electrodes and a dielectric or insulating barrier material for plasma ignition in the elongate cavity.

According to an embodiment of the present invention, the dielectric may be a tubular dielectric defining the elongate cavity therein, and the first and second electrodes may be arranged on an outer surface of the tubular dielectric to be spaced apart from each other.

According to another embodiment of the present invention, the dielectric may be a tubular or bulk dielectric defining the elongate cavity therein, and the first and second electrodes may be formed by printing a metallic material on an outer surface of the dielectric.

According to a further embodiment of the present invention, the first and second electrodes may be shaped to define the elongate cavity when the first and second electrodes mate with each other with an insulating material or dielectric material interposed therebetween.

According to a still further embodiment of the present invention, the first electrode may be a metallic tube defining the elongate cavity, the second electrode may be a metallic rod positioned within the elongate cavity to be spaced apart from the first electrode, and the dielectric is coated or installed to an inner surface of the first electrode or an outer surface of the second electrode.

Preferably, a power source connected to the first electrode or the second electrode may be a medium frequency (MF) power source.

Preferably, the nozzle may be configured to be detached and remounted, i.e., to be replaceable.

The plasma gun according to an embodiment of the present invention may be configured so that a pulse power source or a sinusoidal wave type power source of low peak current is used or on-off timing of a power source is adjusted to control plasma discharge temperature.

According to another aspect of the present invention, there is provided a plasma gun for medical treatment, wherein electrodes for generating plasma are included in an elongate cavity communicating with a nozzle, a dielectric is positioned adjacent to at least one of the electrodes, and a medium frequency (MF) power source is used as a power source for supplying high voltage power to at least one of the electrodes.

### BRIEF DESCRIPTION OF THE DRAWINGS

FIG. 1 is a view illustrating a plasma gun for medical treatment according to an embodiment of the present invention.

FIG. 2 is a sectional view of the plasma gun for medical treatment shown in FIG. 1, illustrating a plasma discharge unit according to the embodiment of the present invention.

FIG. 3 is a view illustrating a plasma discharge unit of a plasma gun for medical treatment according to another embodiment of the present invention.

FIG. 4 is a view illustrating a plasma discharge unit of a plasma gun for medical treatment according to a further embodiment of the present invention.

FIGS. 5a, 5b and 5c are views illustrating plasma discharge units of plasma guns for medical treatment according to still further embodiments of the present invention.

FIGS. 6(a) and (b) are views illustrating replaceable nozzles of plasma guns for medical treatment according to still further embodiments of the present invention.

### DETAILED DESCRIPTION OF ILLUSTRATIVE EMBODIMENTS

Hereinafter, preferred embodiments of the present invention will be described in detail with reference to the accompanying drawings. The following embodiments are provided only for illustrative purposes so that those skilled in the art can fully understand the spirit of the present invention. Therefore, the present invention is not limited to the following embodiments but may be implemented in other forms. In the drawings, the widths, lengths, thicknesses and the like of elements may be exaggerated for convenience of illustration. Like reference numerals indicate like elements throughout the specification and drawings.

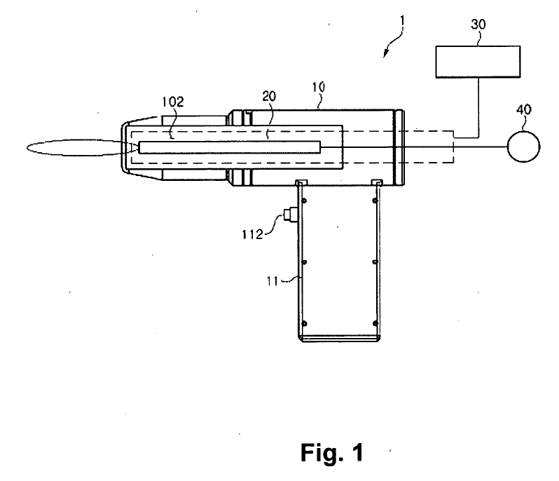

FIG. 1 is a view illustrating a plasma gun for medical treatment according to an embodiment of the present invention.

Referring to FIG. 1, a plasma gun 1 for medical treatment of this embodiment includes an elongate housing 10 having a knob 11 attached to a lower portion thereof. The elongate housing 10 has an elongate chamber 102 defined therein, and a nozzle for spraying plasma generated as described below is positioned at an end of the chamber 102.

In addition, the plasma gun 1 for medical treatment further includes a plasma discharge unit 20, which has electrodes and a dielectric or insulating barrier material and provided in the elongate chamber 102. The plasma discharge unit 20 is to generate stable and reliable atmospheric plasma using dielectric barrier discharge (i.e., DBD) under the atmospheric pressure condition, and may be maintained to be fixed in the elongate chamber 102 by any holder (not shown) arranged in the elongate chamber 102.

Also, the plasma gun 1 for medical treatment further includes a gas supply unit 30 for supplying a reaction gas into the elongate chamber 102 and a power source 40 for applying high voltage to at least one of the electrodes of the plasma discharge unit 20. The gas supply unit 30 allows the reaction gas to flow in the elongate chamber 102 at large pressure enough to provide spraying force to the plasma generated as described below.

According to a preferred embodiment of the present invention, the power source 40 supplies the electrodes of the plasma discharge unit 20 with medium frequency (MF) power whose the frequency ranges from some tens of kHz to some hundreds of kHz. In connection with the foregoing, the gas supply unit 30 may supply the elongate chamber 102 with various kinds of reaction gases (or process gases) such as O₂ and CO₂ as well as He.

Meanwhile, the knob 11 is provided with a switch 112 with which a user may selectively turns on/off the power source 40 for supplying the plasma discharge unit 20 with high voltage power. The plasma gun for medical treatment may have a handheld configuration having a knob, as shown in FIG. 1, or the configuration which may be mounted to a robot arm although not shown.

In the meantime, the plasma gun 1 for medical treatment may adjust the plasma temperature in a broader range by using a pulse power source or a sinusoidal wave type power source of low peak current and/or a regulator for adjusting on/off timing of the high voltage power. Such adjustment of the plasma temperature allows the plasma gun 1 for medical treatment to be broadly applied for various medical applications for various biological tissues.

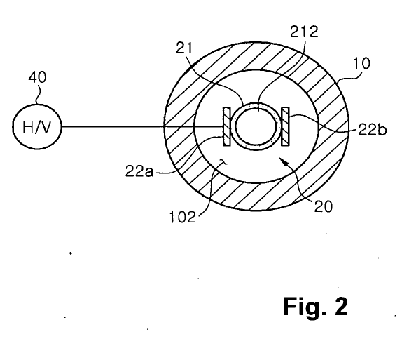

FIG. 2 is a sectional view illustrating in more detail the configuration of the plasma discharge unit provided within the plasma gun shown in FIG. 1.

Referring to FIG. 2, the plasma discharge unit 20 includes a hollow cylindrical dielectric 21 having an elongate cavity 212 provided therein, and first and second electrodes 22a and 22b made of metal which are arranged to face each other and to be spaced apart from each other on an outer surface of the dielectric 21.

The first and second electrodes 22a and 22b of metallic plates are installed to face each other and to be spaced apart from each other on the outer surface of the hollow cylindrical dielectric 21. At this time, the first electrode 22a is connected to the MF power source 40, thereby being supplied with the high voltage power from the MF power source 40, while the second electrode 22b serves as a ground electrode. Alternatively, instead of using one of the electrodes as the ground electrode, the first and second electrodes 22a and 22b may serve as relative electrodes which respectively have positive and negative polarities. The hollow cylindrical dielectric 21 may be formed of an insulating material such as ceramic or polymer.

If the high voltage power from the MF power source 40 is applied to the first electrode 22a and/or the second electrode 22b, the dielectric barrier discharge causes the reaction gas to be stably plasma-ignited in the elongate cavity 212 in the dielectric, and the generated plasma is sprayed to the outside through an end of the elongate cavity 212, i.e., a plasma ejection hole. At this time, the shape and area of the plasma to be sprayed may be adjusted depending on the shape of a spraying nozzle adjacent to the ejection hole.

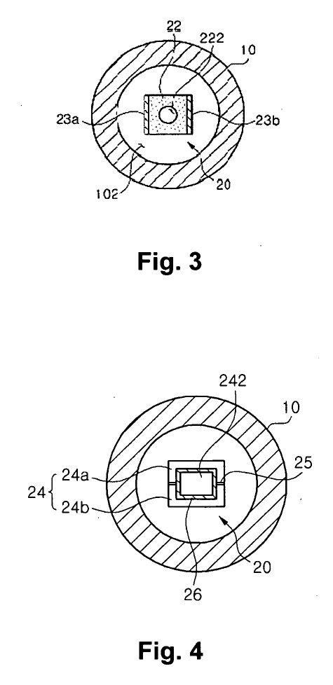

FIG. 3 is a sectional view illustrating the configuration of a plasma discharge unit according to another embodiment of the present invention.

Referring to FIG. 3, the plasma discharge unit 20 according to this embodiment includes a bulk dielectric 22 having an elongate cavity 222 bored through the central region thereof. Further, the bulk dielectric 22 has a cross section of a substantially quadrangular shape, and includes first and second patterned electrodes 23a and 23b which are formed by printing a conductive metal material on both opposite surfaces. As in the previous embodiment, if the high voltage from an MF power source is applied to the first and second patterned electrode 23a and 23b, the dielectric barrier discharge allows stable and reliable plasma to be generated in the elongate cavity 222 through which the reaction gas flows. Further, the generated plasma is sprayed to the outside through a plasma ejection hole positioned at an end of the elongate cavity 222 and a nozzle continued to the plasma ejection hole. At this time, the bulk dielectric 22 is preferably made of ceramic or polymer. The dielectric having a cross section of a rectangular shape is shown in the figure but there is no limitation concerning the sectional shape of the bulk dielectric 22 if an elongate cavity is formed therein.

FIG. 4 is a sectional view illustrating the configuration of a plasma discharge unit according to a further embodiment of the present invention.

Referring to FIG. 4, the plasma discharge unit 20 according to this embodiment includes a tubular member 24 which is made of an elongate metal and has an elongate cavity 242 formed therethrough. Further, the tubular member 24 includes first and second channel-shaped electrodes 24a and 24b which mate with each other to define the elongate cavity 242. The first and second channel-shaped electrodes 24a and 24b are insulated from each other by a dielectric material or insulating material 25 interposed therebetween. At this time, the insulating material 25 may be an adhesive for providing adhesion to a portion where the first and second channel-shaped electrodes 24a and 24b mate with each other. Further, a dielectric 26 for dielectric barrier discharge is partially or entirely formed on an inner surface of the tubular member 24.

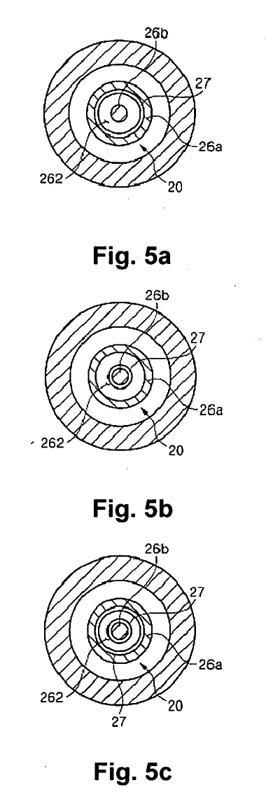

FIGS. 5a to 5c are sectional views illustrating the configuration of plasma discharge units according to still further embodiments of the present invention.

In the plasma discharge units shown in FIGS. 5a to 5c, a first electrode 26a is made of a metallic tube for defining an elongate cavity 262, and a second electrode 26b is made of a metallic rod positioned in the elongate cavity 262 to be spaced apart from the first electrode 26a. A dielectric 27 may be formed on an inner surface of the first electrode 26a (as shown FIG. 5a), an outer surface of the second electrode 26b (as shown in FIG. 5b), or both of the inner surface of the first electrode 26a and the outer surface of the second electrode 26b (as shown in FIG. 5c). At this time, the dielectric is formed on the first electrode 26a and/or the second electrode 26b by a coating or attaching process.

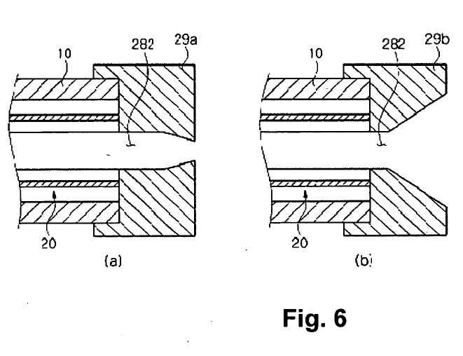

FIGS. 6(a) and (b) are sectional views illustrating the configuration of nozzles of plasma guns for medical treatment according to embodiments of the present invention.

Referring to FIGS. 6(a) and (b), a nozzle 29a or 29b is configured to be replaceably detached and remounted and to communicate with an end of an elongate cavity 282, i.e., a plasma ejection hole, formed on the plasma discharge unit 20 which includes the electrodes and the dielectric. For the purpose of the detachment and remounting, the nozzle 29a or 29b is coupled adjacent to the ejection hole of the plasma gun, for example, in a screwing or hooking manner. FIG. 6(a) shows the configuration of the nozzle 29a for spraying plasma to be converged to a minute dimension, while FIG. 6(b) shows the configuration of the nozzle 29b for spraying the plasma to be broadly diffused. Instead of the function or shape of the nozzles as shown in FIGS. 6(a) and (b), nozzles with various functions and shapes may be replaceably used.

The present invention may be broadly used in various medical applications, as compared with conventional applications, for example, healing for injuries, sterilization for biological tissues, treatment for blood coagulation, healing for skin tumors, healing for bone tumors, fat management and treatment, and skin management and operation. The plasma gun for medical treatment according to the present invention has advantages in that the improved configuration of the chamber for supplying the reaction gas and the plasma discharge unit independently positioned within the chamber causes the plasma to be stably and reliably generated, so that the generated plasma can be preferably used as medial applications for biological tissues, and the application range of the plasma gun for medical treatment can be increased by employing various nozzles capable of adjusting the area (and shape) of the plasma. Further, the present invention has an advantage in that the application range of the plasma gun for medical treatment can be increased by adjusting the temperature of the plasma. Furthermore, the efficiency of the plasma gun can be more improved by employing the configuration in which the flow of the reaction gas is changed into a tangential flow or a laminar flow.

What is claimed:

1. A plasma gun for medical treatment using atmospheric plasma, comprising:
a housing having an elongate chamber provided therein, the chamber having an end at which a nozzle for spraying plasma is positioned;
a gas supply unit for supplying a reaction gas to the chamber; and
a plasma discharge unit formed with an elongate cavity communicating with the nozzle, the plasma discharge unit including first and second electrodes and a dielectric or insulating barrier material for plasma ignition in the elongate cavity.

2. The plasma gun as claimed in claim 1 wherein the dielectric is a tubular dielectric defining the elongate cavity therein, and the first and second electrodes are arranged on an outer surface of the tubular dielectric to be spaced apart from each other.

3. The plasma gun as claimed in claim 1 wherein the dielectric is a tubular or bulk dielectric defining the elongate cavity therein, and the first and second electrodes are formed by printing a metallic material on an outer surface of the dielectric.

4. The plasma gun as claimed in claim 1 wherein the first and second electrodes are shaped to define the elongate cavity when the first and second electrodes mate with each other with an insulating material or dielectric material interposed therebetween.

5. The plasma gun as claimed in claim 1 wherein the first electrode is a metallic tube defining the elongate cavity, the second electrode is a metallic rod positioned within the elongate cavity to be spaced apart from the first electrode, and the dielectric is coated or installed to an inner surface of the first electrode or an outer surface of the second electrode.

6. The plasma gun as claimed in claim 1 wherein a power source connected to the first electrode or the second electrode is a medium frequency (MF) power source.

7. The plasma gun as claimed in claim 1 wherein the nozzle is replaceable.

8. The plasma gun as claimed in claim 1 wherein a pulse power source or a sinusoidal wave type power source of low peak current is used or on-off timing of a power source is adjusted to control plasma discharge temperature.

9. A plasma gun for medical treatment,
wherein electrodes for generating plasma are included in an elongate cavity communicating with a nozzle, a dielectric is positioned adjacent to at least one of the electrodes, and a medium frequency (MF) power source is used as a power source for supplying high voltage power to at least one of the electrodes.
